(19) United States
(12) Patent Application Publication
(10) Pub. No.: US 2025/0091993 A1
(43) Pub. Date: Mar. 20, 2025

(54) SALTS AND SOLID FORMS OF N-ETHYL-2-(5-FLUORO-1H-INDOL-3-YL)-N-METHYLETHAN-1-AMINE

(71) Applicant: Terran Biosciences Inc., Miami Beach, FL (US)

(72) Inventors: Matthew DUNCTON, Las Vegas, NV (US); Samuel CLARK, Miami, FL (US)

(21) Appl. No.: 18/832,754

(22) PCT Filed: Jan. 26, 2023
(86) PCT No.: PCT/US2023/061382

§ 371 Date: Jul. 24, 2024

Related U.S. Application Data
(60) Provisional application No. 63/367,449, filed on Jun. 30, 2022.
(60) Provisional application No. 63/303,855, filed on Jan. 27, 2022.

(51) Int. Cl. C07D 209/16; A61K 31/36; A61K 31/4045; A61K 45/06

(52) U.S. Cl. CPC C07D 209/16; A61K 31/36; A61K 31/4045; A61K 45/06

(57) ABSTRACT

Disclosed herein are salt and solid forms of N-ethyl-2-(5-fluoro-1H-indol-3-yl)-N-methylethan-1-amine. The solid form may be a salt and/or a crystalline form of V-ethyl-2-(5-fluoro-1H-indol-3-yl)-N-methylethan-1-amine, such as a polymorph of N-eth-yl-2-(5-fluoro-1H-indol-3-yl)-N-methylethan-1-amine or a salt thereof. Also disclosed are methods for making the salts and solid forms and methods for administering the same. The solid forms of V-ethyl-2-(5-fluoro-1H-indol-3-yl)-N-methylethan-1-amine are useful for treating neurological disease and/or a psychiatric disorder in a subject.

### CROSS-REFERENCE TO RELATED APPLICATIONS

This application claims priority to and the benefit of U.S. Provisional Application No. 63/303,855, filed on Jan. 27, 2022, and 63/367,449, filed on Jun. 30, 2022, which are incorporated by reference herein in their entirety for all purposes.

### SUMMARY

Disclosed herein are forms of N-ethyl-2-(5-fluoro-1H-indol-3-yl)-N-methylethan-1-amine, including solid forms of N-ethyl-2-(5-fluoro-1H-indol-3-yl)-N-methylethan-1-amine, salts of N-ethyl-2-(5-fluoro-1H-indol-3-yl)-N-methylethan-1-amine and solid forms thereof, including crystalline forms of N-ethyl-2-(5-fluoro-1H-indol-3-yl)-N-methylethan-1-amine and salts thereof, as well as polymorphs of solid forms of N-ethyl-2-(5-fluoro-1H-indol-3-yl)-N-methylethan-1-amine.

Also disclosed are methods for making the salt and solid forms of N-ethyl-2-(5-fluoro-1H-indol-3-yl)-N-methylethan-1-amine and methods for using the salt and solid forms of N-ethyl-2-(5-fluoro-1H-indol-3-yl)-N-methylethan-1-amine. In some embodiments, the solid form of N-ethyl-2-(5-fluoro-1H-indol-3-yl)-N-methylethan-1-amine is a form of the free base form of the compound, such as polymorph of the free base form of the compound. In other embodiments, the solid form of N-ethyl-2-(5-fluoro-1H-indol-3-yl)-N-methylethan-1-amine is a salt, and may be a polymorph of the salt. In some embodiments, a N-ethyl-2-(5-fluoro-1H-indol-3-yl)-N-methylethan-1-amine salt of the present disclosure need not be in solid form. The salt may be formed from an acid selected from hydrochloric acid, fumaric acid, galactaric (mucic) acid, naphthalene-1,5-disulfonic acid, citric acid, sulfuric acid, d-glucuronic acid, ethane-1,2-disulfonic acid, lactobionic acid, p-toluenesulfonic acid, D-glucoheptonic acid, thiocyanic acid, (−)-L-pyroglutamic acid, methanesulfonic acid, L-malic acid, dodecylsulfuric acid, hippuric acid, naphthalene-2-sulfonic acid, D-gluconic acid, benzenesulfonic acid, D,L-lactic acid, oxalic acid, oleic acid, glycerophosphoric acid, succinic acid, ethanesulfonic acid 2-hydroxy, glutaric acid, L-aspartic acid, cinnamic acid, maleic acid, adipic acid, phosphoric acid, sebacic acid, ethanesulfonic acid, (+)-camphoric acid, glutamic acid, acetic acid, xinafoic acid, or a combination thereof. In any embodiments, a stoichiometric ratio of acid to N-ethyl-2-(5-fluoro-1H-indol-3-yl)-N-methylethan-1-amine is from 0.4 to 2.2, such as from 0.5 to 2, or from 0.5, 1 or 2.

In any embodiments, the solid form may be a crystalline solid, a hydrate, or a combination thereof. The crystalline solid may be substantially a single form, such as a polymorph form. And the polymorph may be selected to have one or more desired properties, particularly improved properties, such as physical properties, chemical properties, pharmacokinetic properties, or a combination thereof. The one or more desired properties may comprise melting point, glass transition temperature, flowability, thermal stability, mechanical stability, shelf life, stability against polymorphic transition, hygroscopic properties, solubility in water and/or organic solvents, reactivity, compatibility with excipients and/or delivery vehicles, bioavailability, absorption, distribution, metabolism, excretion, toxicity including cytotoxicity, dissolution rate, half-life, or a combination thereof.

Also disclosed herein are embodiments, of a pharmaceutical composition, comprising a salt and/or a solid form of N-ethyl-2-(5-fluoro-1H-indol-3-yl)-N-methylethan-1-amine, and a pharmaceutically acceptable excipient.

A method for administering salts and solid forms of N-ethyl-2-(5-fluoro-1H-indol-3-yl)-N-methylethan-1-amine also is disclosed herein. In some embodiments, the method comprises administering to a subject an effective amount of a solid form of N-ethyl-2-(5-fluoro-1H-indol-3-yl)-N-methylethan-1-amine, or a pharmaceutical composition thereof. In some embodiments, the subject is suffering from a neurological disease or a psychiatric disorder, or both, such as a neurodegenerative disorder. The neurological disorder or psychiatric disorder, or both, may comprise depression, addiction, anxiety, or a post-traumatic stress disorder, and/or the neurological disorder or psychiatric disorder, or both, may comprise treatment resistant depression, suicidal ideation, major depressive disorder, bipolar disorder, schizophrenia, or substance use disorder. In some embodiments, the neurological disorder or psychiatric disorder, or both, comprises stroke, traumatic brain injury, or a combination thereof.

The method may comprise further comprising administering an effective amount of an empathogenic agent and/or a 5-HT2A antagonist to the subject. The 5-HT2A antagonist may be selected from MDL-11,939. The 5-HT2A antagonist may be selected from glemanserin (MDL-11,939), eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), pimavanserin (ACO-103), pruvanserin, nelotanserin, lorcaserin, flibanserin, roluperiodone or a pharmaceutically acceptable salt, solvate, metabolite, deuterated analogue, derivative, prodrug, or combinations thereof. In some embodiments, the serotonin receptor modulator comprises eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), pimavanserin, olanzapine, risperidone, nelotanserin and lorcaserin.

In any embodiments, administering the compound form, including a salt and/or solid form of the compound comprises oral, parenteral, or topical administration. In certain embodiments, oral administration is used, but in other particular embodiments, administration is by injection, inhalation, intraocular, intravaginal, intrarectal or transdermal routes.

The foregoing and other objects, features, and advantages of the present disclosure will become more apparent from the following detailed description, which proceeds with reference to the accompanying figures.

### BRIEF DESCRIPTION OF THE FIGURES

FIG. 1 provides an XRPD diffractogram of N-ethyl-2-(5-fluoro-1H-indol-3-yl)-N-methylethan-1-amine·HCl.

FIG. 2 provides an expanded section of the XRPD diffractogram of FIG. 1.

FIG. 3 provides an expanded section of the XRPD diffractogram of FIG. 1

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

“Administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“N-ethyl-2-(5-fluoro-1H-indol-3-yl)-N-methylethan-1-amine” refers to the compound

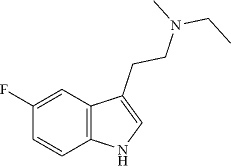 [SMILES: CCN(C)CCc1c[nH]c2ccc(F)cc12]

### N-ethyl-2-(5-fluoro-1H-indol-3-yl)-N-methylethan-1-amine

“Subject” refers to an animal, such as a mammal, including, but not limited to, primates (e.g., humans), cows, sheep, goats, horses, dogs, cats, rabbits, rats, mice and the like. In certain embodiments, the subject is a human subject.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Combination therapy” refers to a method of treating a disease or disorder, wherein two or more different pharmaceutical agents are administered in overlapping regimens so that the subject is simultaneously exposed to both agents. For example, the compounds and compound forms of the disclosure can be used in combination with other pharmaceutically active compounds. The compounds and compound forms of the disclosure can be administered simultaneously (as a single preparation or separate preparation) or sequentially to the other drug therapy. In general, a combination therapy envisions administration of two or more drugs during a single cycle or course of therapy.

“Neurotrophic factors” refers to a family of soluble peptides or proteins which support the survival, growth, and differentiation of developing and mature neurons.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT2A) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT2A agonist” can be used to refer to a compound that exhibits an EC50 with respect to 5HT2A activity of no more than about 100 mM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors and colors. One of skill in the art will recognize that other pharmaceutical excipients are useful in the present disclosure.

### Compounds

Disclosed herein are salts and solid forms of N-ethyl-2-(5-fluoro-1H-indol-3-yl)-N-methylethan-1-amine, which are useful to treat various disorders, such as brain disorders. Also disclosed are methods for making the salts and solid forms of N-ethyl-2-(5-fluoro-1H-indol-3-yl)-N-methylethan-1-amine and methods of administering the salts and solid forms of the compound

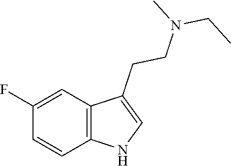 [SMILES: CCN(C)CCc1c[nH]c2ccc(F)cc12]

### N-ethyl-2-(5-fluoro-1H-indol-3-yl)-N-methylethan-1-amine

In some embodiments, a solid form of the compound is a crystalline form of N-ethyl-2-(5-fluoro-1H-indol-3-yl)-N-methylethan-1-amine. In some embodiments, the solid form of N-ethyl-2-(5-fluoro-1H-indol-3-yl)-N-methylethan-1-amine is a polymorph of N-ethyl-2-(5-fluoro-1H-indol-3-yl)-N-methylethan-1-amine, such as a polymorph of the free base compound or a polymorph of the salt. In some embodiments, the solid form of the compound is a salt of the compound. In some embodiments, the solid form of the compound is a crystalline salt form of the compound, such as an acid addition salt form.

N-ethyl-2-(5-fluoro-1H-indol-3-yl)-N-methylethan-1-amine can be made using (4-fluorophenyl) hydrazine hydrochloride as a starting material and converting it to the corresponding 5-fluoro-3-(hydroxyalkyl)-indole intermediate using dihydropyran, H₂SO₄, and dimethylamine. The 5-fluoro-3-(hydroxyalkyl)-indole intermediate is then converted to a mesylate derivative using methanesulfonyl chloride, triethylamine, and CH₂Cl₂, which in turn is converted to N-ethyl-2-(5-fluoro-1H-indol-3-yl)-N-methylethan-1-amine by reacting the mesylate derivative with N-methylethanamine in dioxane at 100° C.

### Salts

In some embodiments, the solid form of N-ethyl-2-(5-fluoro-1H-indol-3-yl)-N-methylethan-1-amine comprises a salt of N-ethyl-2-(5-fluoro-1H-indol-3-yl)-N-methylethan-1-amine. Suitable salts include pharmaceutically acceptable salts of N-ethyl-2-(5-fluoro-1H-indol-3-yl)-N-methylethan-1-amine.

In some embodiments, the salt of N-ethyl-2-(5-fluoro-1H-indol-3-yl)-N-methylethan-1-amine may be formed from a suitable pharmaceutically acceptable acid, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, xinafoic acid and the like.

In other embodiments, the salt of N-ethyl-2-(5-fluoro-1H-indol-3-yl)-N-methylethan-1-amine may be formed from a suitable pharmaceutically acceptable base, including, without limitation, inorganic bases such as sodium, potassium, lithium, ammonium, calcium, magnesium, iron, zinc, copper, manganese, aluminum salts and the like. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Additional information concerning pharmaceutically acceptable salts can be found in, for example, S. M. Berge, et al., “Pharmaceutical Salts,” J. Pharm. Sci., 1977; 66:1-19 which is incorporated herein by reference.

In some embodiments, the salt may be formed using an acid from Table 1.

TABLE 1

| | |
|---|---|
| naphthalene-1,5-disulfonic acid | citric acid |
| sulfuric acid | d-glucuronic acid |
| ethane-1,2-disulfonic acid | lactobionic acid |
| p-toluenesulfonic acid | D-glucoheptonic acid |
| thiocyanic acid | (−)-L-pyroglutamic acid |
| methanesulfonic acid | L-malic acid |
| dodecylsulfuric acid | hippuric acid |
| naphthalene-2-sulfonic acid | D-gluconic acid |
| benzenesulfonic acid | D,L-lactic acid |
| oxalic acid | oleic acid |
| glycerophosphoric acid | succinic acid |
| ethanesulfonic acid, 2-hydroxy | glutaric acid |
| L-aspartic acid | cinnamic acid |
| maleic acid | adipic acid |
| phosphoric acid | sebacic acid |
| ethanesulfonic acid | (+)-camphoric acid |
| glutamic acid | acetic acid |
| pamoic (embonic) acid | nicotinic acid |
| glutaric acid, 2-oxo- | isobutyric acid |
| 2-naphthoic acid, 1-hydroxy | propionic acid |
| malonic acid | lauric acid |
| gentisic acid | stearic acid |
| L-tartaric acid | orotic acid |
| hydrochloric acid | carbonic acid |
| galactaric (mucic) acid | Fumaric acid |

The acid salts of N-ethyl-2-(5-fluoro-1H-indol-3-yl)-N-methylethan-1-amine disclosed herein can have any suitable stoichiometric ratio of acid to N-ethyl-2-(5-fluoro-1H-indol-3-yl)-N-methylethan-1-amine. In one embodiment, the molar ratio of acid to N-ethyl-2-(5-fluoro-1H-indol-3-yl)-N-methylethan-1-amine is from 0.4 to 2.2, such as forms wherein the salt has a stoichiometric ratio of acid to N-ethyl-2-(5-fluoro-1H-indol-3-yl)-N-methylethan-1-amine of from 0.5 to 2, such as 0.5, 1 or 2.

### Solid Forms

Embodiments of N-ethyl-2-(5-fluoro-1H-indol-3-yl)-N-methylethan-1-amine of the present disclosure are in a solid form. The solid form may be a crystalline form or an amorphous form. In some embodiments, the solid form is a crystalline form, such as a polymorph. In some embodiments, the solid form of N-ethyl-2-(5-fluoro-1H-indol-3-yl)-N-methylethan-1-amine is a salt. And in certain embodiments, the solid form is a crystalline salt form of N-ethyl-2-(5-fluoro-1H-indol-3-yl)-N-methylethan-1-amine. A person of ordinary skill in the art understands that solid forms of N-ethyl-2-(5-fluoro-1H-indol-3-yl)-N-methylethan-1-amine, such as crystalline forms including salt and non-salt crystalline forms of N-ethyl-2-(5-fluoro-1H-indol-3-yl)-N-methylethan-1-amine, may exist in more than one crystal form. Such different forms are referred to as polymorphs. In some embodiments, the disclosed compounds are particular polymorphs of N-ethyl-2-(5-fluoro-1H-indol-3-yl)-N-methylethan-1-amine or a N-ethyl-2-(5-fluoro-1H-indol-3-yl)-N-methylethan-1-amine salt.

In some embodiments, the solid form of N-ethyl-2-(5-fluoro-1H-indol-3-yl)-N-methylethan-1-amine disclosed herein is selected to be a crystalline form, such as a particular polymorph of a crystalline form of N-ethyl-2-(5-fluoro-1H-indol-3-yl)-N-methylethan-1-amine that provides one or more desired properties. In one embodiment, the crystalline form offers advantages over the amorphous form of N-ethyl-2-(5-fluoro-1H-indol-3-yl)-N-methylethan-1-amine. In another embodiment, the disclosed polymorph offers improved properties as compared to another polymorph of N-ethyl-2-(5-fluoro-1H-indol-3-yl)-N-methylethan-1-amine. The N-ethyl-2-(5-fluoro-1H-indol-3-yl)-N-methylethan-1-amine may be a salt or free base compound. The one or more desired properties may include, but are not limited to, physical properties, including but not limited to, melting point, glass transition temperature, flowability, and/or stability, such as thermal stability, mechanical stability, shelf life, stability against polymorphic transition, etc.; chemical properties, such as, but not limited to, hygroscopic properties, solubility in water and/or organic solvents, reactivity, compatibility with excipients and/or delivery vehicles; and/or pharmacokinetic properties, such as, but not limited to, bioavailability, absorption, distribution, metabolism, excretion, toxicity including cytotoxicity, dissolution rate, and/or half-life.

The desired polymorph may be produced by techniques known to persons of ordinary skill in the art with the benefit of the present disclosure. Such techniques include, but are not limited to, crystallization in particular solvents and/or at particular temperatures, supersaturation, using a precipitation agent, such as a salt, glycol, alcohol, etc., co-crystallization, lyophilization, spray drying, freeze drying, and/or complexing with an inert agent.

Techniques to identify a particular solid form of N-ethyl-2-(5-fluoro-1H-indol-3-yl)-N-methylethan-1-amine are known to persons of ordinary skill in the art with the benefit of the present disclosure, and include, but are not limited to, X-ray crystallography, X-ray diffraction, electron crystallography, powder diffraction, including X-ray, neutron, or electron diffraction, X-ray fiber diffraction, small-angle X-ray scattering, and/or melting point.

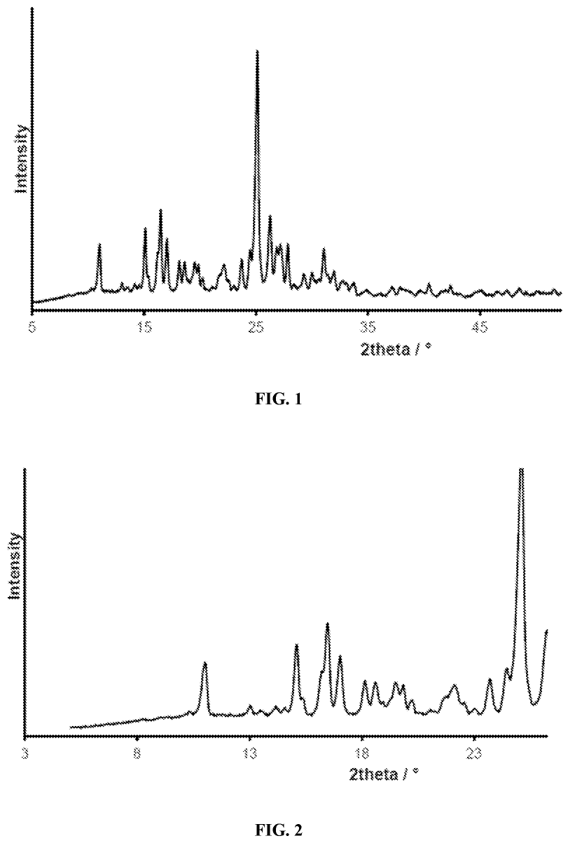

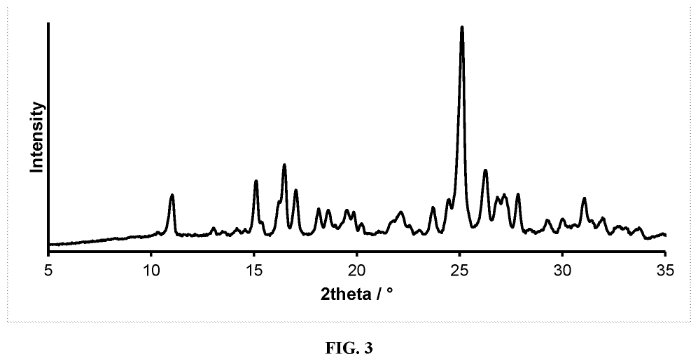

In some embodiments, the present disclosure provides solid forms of N-ethyl-2-(5-fluoro-1H-indol-3-yl)-N-methylethan-1-amine hydrochloride, e.g., crystalline forms of N-ethyl-2-(5-fluoro-1H-indol-3-yl)-N-methylethan-1-amine hydrochloride. In some embodiments, the N-ethyl-2-(5-fluoro-1H-indol-3-yl)-N-methylethan-1-amine hydrochloride XRPD profile is substantially similar to that shown in FIG. 1.

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by an XRPD signal at 25.1 °2θ (±0.2°2θ; ±0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °20 and 16.5 °2θ (±0.2 °2θ; ±0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three XRPD signals selected from the group consisting of 25.1 °2θ, 16.5 °2θ, and 26.2 °2θ (±0.2 °2θ; +0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °2θ, 16.5 °2θ, and 26.2 °2θ (±0.2 °2θ; ±0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three or more XRPD signals selected from the group consisting of 25.1 °θ, 16.5 °2θ, 26.2 °2θ, and 15.1 °2θ (±0.2 °2θ; ±0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °2θ, 16.5 °2θ, 26.2 °2θ, and 15.1 °2θ (±0.2 °2θ; ±0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three or more XRPD signals selected from the group consisting of 25.1 °θ, 16.5 °2θ, 26.2 °2θ, 15.1 °2θ, and 17.0 °2θ (±0.2 °2θ; ±0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °2θ, 16.5 °2θ, 26.2 °2θ, 15.1 °2θ, and 17.0 °2θ (±0.2 °2θ; ±0.1 °2θ; or ±0.0 °2θ: Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three or more XRPD signals selected from the group consisting of 25.1 °2θ, 16.5 °2θ, 26.2 °2θ, 15.1 °θ, 17.0 °2θ, and 11.0 °2θ (±0.2 °2θ; ±0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °θ, 16.5 °2θ, 26.2 °2θ, 15.1 °θ, 17.0 °2θ, and 11.0 °2θ (±0.2 °2θ; ±0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three or more XRPD signals selected from the group consisting of 25.1 °θ, 16.5 °2θ, 26.2 °2θ, 15.1 °2θ, 17.0 °2θ, 11.0 °2θ, and 27.8 °2θ (±0.2 °2θ; ±0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °θ, 16.5 °2θ, 26.2 °2θ, 15.1 °2θ, 17.0 °2θ, 11.0 °2θ, and 27.8 °2θ (±0.2 °2θ; =0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three or more XRPD signals selected from the group consisting of 25.1 °θ, 16.5 °2θ, 26.2 °2θ, 15.1 °2θ, 17.0 °2θ, 11.0 °2θ, 27.8 °2θ, and 27.2 °2θ (±0.2 °2θ; ±0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °θ, 16.5 °2θ, 26.2 °2θ, 15.1 °θ, 17.0 °2θ, 11.0 °2θ, 27.8 °2θ, and 27.2 °2θ (±0.2 °2θ; ±0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three or more XRPD signals selected from the group consisting of 25.1 °θ, 16.5 °2θ, 26.2 °2θ, 15.1 °θ, 17.0 °2θ, 11.0 °2θ, 27.8 °2θ, 27.2 °2θ, and 31.0 °2θ (±0.2 °2θ; ±0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °2θ, 16.5 °2θ, 26.2 °2θ, 15.1 °2θ, 17.0 °2θ, 11.0 °2θ, 27.8 °2θ, 27.2 °2θ, and 31.0 °2θ (±0.2 °2θ; ±0.1 °2θ; or ±0.0 °2θ; Cu Kα1 radiation).

In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by two or more, or three or more XRPD signals selected from the group consisting of 25.1 °θ, 16.5 °2θ, 26.2 °2θ, 15.1 °θ, 17.0 °2θ, 11.0 °2θ, 27.8 °2θ, 27.2 °2θ, 31.0 °2θ, and 26.8 °2θ (±0.2 °2θ; ±0.1 °2θ; or ±0.0 °2θ; Cu Kα1 radiation). In some embodiments, the solid form of N-ethyl-2-(5-fluoro-1H-indol-3-yl)-N-methylethan-1-amine hydrochloride is crystalline N-ethyl-2-(5-fluoro-1H-indol-3-yl)-N-methylethan-1-amine hydrochloride characterized by XRPD signals at 25.1 °2θ, 16.5 °2θ, 26.2 °2θ, 15.1 °2θ, 17.0 °2θ, 11.0 °2θ, 27.8 °2θ, 27.2 °2θ, 31.0 °2θ, and 26.8 °2θ (±0.2 °2θ; ±0.1 °2θ; or ±0.0 °2θ; Cu Kα1 radiation).

In some embodiments, the crystalline N-ethyl-2-(5-fluoro-1H-indol-3-yl)-N-methylethan-1-amine hydrochloride is characterized by one, two, three, four, five, six, seven, eight, nine, ten, eleven, twelve, thirteen, fourteen, fifteen, sixteen, seventeen, or eighteen XRPD signals selected from those set forth in Table 1A.

TABLE 1A
Peak list for N-ethyl-2-(5-fluoro-1H-indol-
3-yl)-N-methylethan-1-amine hydrochloride

| No. | Pos. [°2θ] | d-spacing [Å] | Rel. Int. [%] |
|---|---|---|---|
| 1 | 11.0 | 8.1 | 21.0 |
| 2 | 15.1 | 5.9 | 27.5 |
| 3 | 15.4 | 5.8 | 4.2 |
| 4 | 16.2 | 5.5 | 14.0 |
| 5 | 16.5 | 5.4 | 34.3 |
| 6 | 17.0 | 5.2 | 21.5 |
| 7 | 18.1 | 4.9 | 11.7 |
| 8 | 18.6 | 4.8 | 11.9 |
| 9 | 19.5 | 4.6 | 10.8 |
| 10 | 22.1 | 4.0 | 11.1 |
| 11 | 23.7 | 3.8 | 12.5 |
| 12 | 24.5 | 3.6 | 13.4 |
| 13 | 25.1 | 3.5 | 100.0 |
| 14 | 26.2 | 3.4 | 31.6 |
| 15 | 26.8 | 3.3 | 16.1 |
| 16 | 27.2 | 3.3 | 18.2 |
| 17 | 27.8 | 3.2 | 20.5 |
| 18 | 31.0 | 2.9 | 17.5 |

### Pharmaceutical Compositions and Formulations

In some embodiments, the present disclosure provides a pharmaceutical composition comprising one or more of the salts and/or solid forms of N-ethyl-2-(5-fluoro-1H-indol-3-yl)-N-methylethan-1-amine, illustrated above, and a pharmaceutically acceptable excipient. Such compositions are suitable for administration to a subject, such as a human subject.

The presently disclosed pharmaceutical compositions can be prepared in a wide variety of oral, parenteral and topical dosage forms. Oral preparations include tablets, pills, powder, capsules, lozenges, cachets, slurries, suspensions, etc., suitable for ingestion by the patient. The compositions of the present disclosure can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present disclosure can be administered transdermally. The compositions of this disclosure can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present disclosure also provides pharmaceutical compositions including a pharmaceutically acceptable carrier or excipient and the solid form of N-ethyl-2-(5-fluoro-1H-indol-3-yl)-N-methylethan-1-amine of the present disclosure.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% to 70% or 10% to 70% of the compounds of the present disclosure.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present disclosure are dispersed homogeneously therein, as by stirring. The molten homogeneous mixture is then poured into convenient sized molds, allowed to cool, and thereby to solidify.

Liquid form preparations include suspensions, for example, water or water/propylene glycol suspensions.

Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxy cetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxy benzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include suspensions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present disclosure in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the disclosure can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soy bean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present disclosure can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present disclosure can be formulated for parenteral administration, such as intravenous (IV) administration or administration into a body cavity or lumen of an organ. The formulations for administration will commonly comprise a solution or suspension of the compositions of the present disclosure dissolved or suspended in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions or suspensions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present disclosure can be delivered by the use of liposomes which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### Administration

The compositions of the present disclosure can be administered by any suitable means, including oral, parenteral and topical methods. Transdermal administration methods, by a topical route, can be formulated as applicator sticks, suspensions, creams, ointments, pastes, jellies, paints, powders, and aerosols.

The pharmaceutical preparation is preferably in unit dosage form. In such form the preparation is subdivided into unit doses containing appropriate quantities of the compounds of the present disclosure. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present disclosure can be present in any suitable amount, and can depend on various factors including, but not limited to, weight and age of the subject, state of the disease, and the like as is known to those of ordinary skill in the art. Suitable dosage ranges for the compound disclosed herein include from 0.1 mg to 10,000 mg, or 1 mg to 1000 mg, or 10 mg to 750 mg, or 25 mg to 500 mg, or 50 mg to 250 mg. Suitable dosages for the compound of the present disclosure include 1 mg, 5, 10, 20, 30, 40, 50, 60, 70, 80, 90, 100, 200, 300, 400, 500, 600, 700, 800, 900 or 1000 mg.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When a compound of the present disclosure is administered more than once a day, representative intervals include 5, 10, 15, 20, 30, 45 and 60 minutes, as well as 1, 2, 4, 6, 8, 10, 12, 16, 20, and 24 hours. The compounds of the present disclosure can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present disclosure can be co-administered with a second active agent. Co-administration includes administering a compound of the present disclosure and active agent within 0.5, 1, 2, 4, 6, 8, 10, 12, 16, 20, or 24 hours of each other. Co-administration also includes administering a compound of the present disclosure and active agent simultaneously, approximately simultaneously (e.g., within 1, 5, 10, 15, 20, or 30 minutes of each other), or sequentially in any order. Moreover, the compounds of the present disclosure and the active agent can each be administered once a day, or two, three, or more times per day so as to provide the preferred dosage level per day.

The compounds of the present disclosure can be co-administered with a second active agent. In some embodiments, co-administration can be accomplished by co-formulation, such as by preparing a single pharmaceutical composition including both the compounds of the present disclosure and a second active agent. In other embodiments, the compounds of the present disclosure and the second active agent can be formulated separately.

The disclosed compounds and the second active agent can be present in the compositions of the present disclosure in any suitable weight ratio, such as from 1:100 to 100:1 (w/w), or 1:50 to 50:1, or 1:25 to 25:1, or 1:10 to 10:1, or 1:5 to 5:1 (w/w). The compounds of the present disclosure and the second active agent can be present in any suitable weight ratio, such as 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compounds of the present disclosure and the active agent are suitable in the compositions and methods disclosed herein.

### Methods of Treatment

The salts and solid forms of N-ethyl-2-(5-fluoro-1H-indol-3-yl)-N-methylethan-1-amine of the present disclosure can be used for increasing neuronal plasticity. The compounds of the present disclosure can also be used to treat any brain disease. The compounds of the present disclosure can also be used for increasing at least one of translation, transcription or secretion of neurotrophic factors.

In some embodiments, a compound of the present disclosure is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound form of the present disclosure is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the present disclosure provides a method of treating a disease, including administering to a subject in need thereof, a therapeutically effective amount of a compound of the present disclosure. In some embodiments, the disease is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause.

In some embodiments, the N-ethyl-2-(5-fluoro-1H-indol-3-yl)-N-methylethan-1-amine forms of the present disclosure have activity as 5-HT_{2A} modulators. In some embodiments, the compounds of the present disclosure elicit a biological response by activating the 5-HT_{2A} receptor (by way of example, through allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present disclosure are 5-HT_{2A} modulators and promote neural plasticity (e.g., cortical structural plasticity). In some embodiments, the compounds of the present disclosure are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the N-ethyl-2-(5-fluoro-1H-indol-3-yl)-N-methylethan-1-amine forms disclosed function as 5-HT_{2A} modulators (e.g., 5-HT_{2A} agonists) that are non-hallucinogenic or are administered at a non-hallucinogenic dose. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present disclosure to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl) piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl)piperazin-1-yl)propyl)-5-methyl-5-phenylimidazolidine-2,4-dione, 3-(3-(4-(3-Fluorophenyl)piperazin-1-yl)propyl)-5-methyl-5-phenylimidazolidine-2,4-dione, 3-(3-(4-(4-Fluorophenyl)piperazin-1-yl)propyl)-5-methyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-Fluorobenzoyl)piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl)piperazin-1-yl)propyl)-8-phenyl-1,3-diazaspiro[4.5]decane-2,4-dione, 3-(3-(4-(3-Fluorophenyl)piperazin-1-yl)propyl)-8-phenyl-1,3-diazaspiro[4.5]decane-2,4-dione, 3-(3-(4-(4-Fluorophenyl)piperazin-1-yl)propyl)-8-phenyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), pimavanserin, olanzapine, risperidone, nelotanserin and lorcaserin. In some embodiments, the serotonin receptor modulator used as a second therapeutic is pimavanserin or a pharmaceutically acceptable salt, solvate, metabolite, derivative, or prodrug thereof.

In certain embodiments the serotonin receptor modulator is selected from the group consisting of altanserin, blonanserin, eplivanserin, glemanserin, volinanserin, ketanserin, ritanserin, pimavanserin, nelotanserin, pruvanserin, and flibanserin. In one embodiment, the serotonin receptor modulator is selected from the group consisting of eplivanserin, volinanserin, ketanserin, ritanserin, pimavanserin, nelotanserin, pruvanserin, flibanserin, olanzapine, quetiapine, risperidone, and buspirone.

In some embodiments, the serotonin receptor modulator is ketanserin or a pharmaceutically acceptable salt, solvate, metabolite, deuterated analog, derivative, or prodrug thereof. In some embodiments, the serotonin receptor modulator is pimavanserin or a pharmaceutically acceptable salt, solvate, metabolite, deuterated analog, derivative, or prodrug thereof. In some embodiments, the serotonin receptor modulator is eplivanserin or a pharmaceutically acceptable salt, solvate, metabolite, deuterated analog, derivative, or prodrug thereof. In some embodiments, the serotonin receptor modulator is flibanserin or a pharmaceutically acceptable salt, solvate, metabolite, deuterated analog, derivative, or prodrug thereof. In some embodiments, the serotonin receptor modulator is roluperiodone or a pharmaceutically acceptable salt, solvate, metabolite, deuterated analog, derivative, or prodrug thereof.

In some embodiments, the serotonin receptor modulator is administered prior to a compound disclosed herein, such as three or one hours prior to administration of a compound disclosed herein. In some embodiments, the serotonin receptor modulator is administered at most one hour prior to the presently disclosed compounds. Thus, in some embodiments of combination therapy with the presently disclosed compounds, the second therapeutic agent is a serotonin receptor modulator. In some embodiments the second therapeutic agent serotonin receptor modulator is provided at a dose of from 10 mg to 350 mg. In some embodiments, the serotonin receptor modulator is provided at a dose of from 20 mg to 200 mg. In some embodiments, the serotonin receptor modulator is provided at a dose of from 10 mg to 100 mg. In certain such embodiments, a compound of the present disclosure is provided at a dose of from about 100 μg to about 100 mg, or from about 1 mg to about 20 mg, or from about 15 mg to about 30 mg, and the serotonin receptor modulator is provided at a dose of about 1 mg to about 100 mg.

In some embodiments, the serotonin receptor modulator for use with N-ethyl-2-(5-fluoro-1H-indol-3-yl)-N-methylethan-1-amine salts and solid forms disclosed herein, including those described in Table 5, is eplivanserin, wherein the eplivanserin is administered in about 1 mg to about 40 mg, or about 5 mg to about 10 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is volinanserin, wherein the volinanserin is administered in about 1 mg to about 60 mg, or about 5 mg to about 20 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is ketanserin, wherein the ketanserin is administered in about 10 mg to about 80 mg, about 30 mg to about 50 mg, or about 40 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is ritanserin, wherein the ritanserin is administered in about 1 mg to about 40 mg, or about 2.5 mg to about 10 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is pimavanserin, wherein the pimavanserin is administered in about 1 mg to about 60 mg, or about 17 mg to about 34 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is nelotanserin, wherein the nelotanserin is administered in about 1 mg to about 80 mg, or about 40 mg to about 80 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is pruvanserin, wherein the pruvanserin is administered in about 1 mg to about 40 mg, or about 3 mg to about 10 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is flibanserin, wherein the flibanserin is administered in about 10 mg to about 200 mg, or about 80 mg to about 120 mg, or about 100 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is olanzapine, wherein the olanzapine is administered in about 2.5 mg to about 30 mg, or about 5 mg or about 10 mg, or about 20 mg or about 25 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is quetiapine, wherein the quetiapine is administered in about 25 mg to about 800 mg, or about 50 mg to about 100 mg, or about 150 mg or about 200 mg or about 250 mg or about 300 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is an extended-release of quetiapine, wherein the extended-release of quetiapine is administered in about 50 mg to about 300 mg, or about 50 mg or about 100 mg or about 200 mg, or about 300 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is risperidone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is an extended-release of risperidone including RISPERDAL CONSTA, wherein the extended-release of risperidone is administered in about 12.5 mg, or about 25 mg, or about 37.5 mg, or about 50 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In some embodiments, the serotonin receptor modulator for use with the N-ethyl-2-(5-fluoro-1H-indol-3-yl)-N-methylethan-1-amine salts and solid forms disclosed herein, including those described in Table 5, is buspirone, wherein the buspirone is administered in about 1 mg to about 100 mg, or about 1 mg or about 2 mg, or about 3 mg, or about 4 mg, or about 5 mg, or about 6 mg, or about 7 mg, or about 7.5 mg, or about 10 mg, or about 15 mg, or about 22.5 mg, or about 30 mg, or about 37.5 mg, or about 45 mg, or about 52.5 mg, or about 60 mg, or about 1 mg to about 10 mg, or about 5 mg to about 10 mg, or about 10 mg to about 15 mg, or about 15 mg to about 30 mg, or about 30 mg to about 60 mg, or about 60 mg to about 80 mg, or about 80 mg to about 100 mg, and the N-ethyl-2-(5-fluoro-1H-indol-3-yl)-N-methylethan-1-amine salts and solid forms disclosed herein, including those described in Table 5, are administered from about 100 μg to about 100 mg, or from about 1 mg to about 20 mg, or from about 15 mg to about 30 mg.

In certain embodiments, such as those described above a N-ethyl-2-(5-fluoro-1H-indol-3-yl)-N-methylethan-1-amine form disclosed herein, including those described in Table 5, is co-administered with a serotonin receptor modulator in the same or in separate compositions. In one embodiment, the serotonin receptor modulator is administered prior to the N-ethyl-2-(5-fluoro-1H-indol-3-yl)-N-methylethan-1-amine form disclosed herein, including those described in Table 5. In one embodiment, the N-ethyl-2-(5-fluoro-1H-indol-3-yl)-N-methylethan-1-amine form disclosed herein, including those described in Table 5, is administered in a modified release formulation such that the subject is effectively pretreated with serotonin receptor modulator prior to release of an effective amount of the N-ethyl-2-(5-fluoro-1H-indol-3-yl)-N-methylethan-1-amine. In some embodiments the serotonin receptor modulator is part of a single fixed dose formulation that releases serotonin receptor modulator first followed by the N-ethyl-2-(5-fluoro-1H-indol-3-yl)-N-methylethan-1-amine on two different release profiles. In another embodiment the serotonin receptor modulator is administered first as a single dosage and after a length of time, the N-ethyl-2-(5-fluoro-1H-indol-3-yl)-N-methylethan-1-amine form disclosed herein, including those described in Table 5, is administered as a second dosage separate from the first dosage. Thus, in some embodiments, the serotonin receptor modulator is administered or released from a composition provided herein prior to the administration and/or release of the N-ethyl-2-(5-fluoro-1H-indol-3-yl)-N-methylethan-1-amine form disclosed herein, including those described in Table 5. This allows pretreatment to attenuate activation of the serotonin receptor by the N-ethyl-2-(5-fluoro-1H-indol-3-yl)-N-methylethan-1-amine form disclosed herein, including those described in Table 5.

In some embodiments, the serotonin receptor modulator is administered or released from the composition provided herein to pretreat a subject by at least about 5 minutes, 10 minutes, 20 minutes, 30) minutes, 40) minutes, 50) minutes, 1 hour, 1.25 hours, 1.5 hours, 2 hours, or 3 hours prior to the release of the N-ethyl-2-(5-fluoro-1H-indol-3-yl)-N-methylethan-1-amine form disclosed herein, including those described in Table 5. In some embodiments, the serotonin receptor modulator attenuates the activation of the serotonin receptor when the serotonin receptor modulator is used to pretreat at most about 3 hours, 4 hours, 5 hours, 6 hours, 7 hours, 8 hours, 9 hours, or more than 9 hours prior to the release of N-ethyl-2-(5-fluoro-1H-indol-3-yl)-N-methylethan-1-amine form disclosed herein, including those described in Table 5. In some embodiments, the serotonin receptor modulator attenuates the activation of the serotonin receptor when the serotonin receptor modulator is used to pretreat in a range of about 5 minutes to about 3 hours, about 10 minutes to about 3 hours, about 20 minutes to about 3 hours, about 30) minutes to about 3 hours, about 40) minutes to about 3 hours, about 50) minutes to about 3 hours, about 1 hour to about 3 hours, about 5 minutes to about 2 hours, about 10) minutes to about 2 hours, about 20) minutes to about 2 hours, about 30 minutes to about 2 hours, about 40) minutes to about 2 hours, about 50) minutes to about 2 hours, about 1 hour to about 2 hours, about 5 minutes to about 1 hour, about 10) minutes to about 1 hour, about 20 minutes to about 1 hour, about 30) minutes to about 1 hour, about 40) minutes to about 1 hour, or about 50 minutes to about 1 hour prior to the release of the N-ethyl-2-(5-fluoro-1H-indol-3-yl)-N-methylethan-1-amine form disclosed herein, including those described in Table 5.

In a preferred embodiment, the serotonin receptor modulator is administered at about 1 hour to about 3 hours prior to the administration of the N-ethyl-2-(5-fluoro-1H-indol-3-yl)-N-methylethan-1-amine form disclosed herein, including those described in Table 5.

In some embodiments, the serotonin receptor modulator is eplivanserin, wherein the eplivanserin is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat between at least 30 minutes prior and 360 minutes prior to the release or administration of the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat between at least 60 minutes prior and 360 minutes prior to the release or administration the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat between at least 90 minutes and 240) minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat at least 120 minutes prior to the N-ethyl-2-(5-fluoro-1H-indol-3-yl)-N-methylethan-1-amine form disclosed herein, including those described in Table 5.

In some embodiments, the serotonin receptor modulator is eplivanserin, wherein the eplivanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat at least 210 minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat at least 240 minutes prior to the N-ethyl-2-(5-fluoro-1H-indol-3-yl)-N-methylethan-1-amine form disclosed herein, including those described in Table 5.

In some embodiments, the serotonin receptor modulator is eplivanserin, wherein the eplivanserin is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to pretreat at least 360 minutes prior to administration or release of the N-ethyl-2-(5-fluoro-1H-indol-3-yl)-N-methylethan-1-amine form disclosed herein, including those described in Table 5.

In some preferred embodiments, the serotonin receptor modulator is eplivanserin, wherein eplivanserin is administered to pretreat between about 60 minutes and about 180) minutes prior to the administration or release of the N-ethyl-2-(5-fluoro-1H-indol-3-yl)-N-methylethan-1-amine form disclosed herein, including those described in Table 5.

In some embodiments, the serotonin receptor modulator is volinanserin, wherein the volinanserin is administered to pretreat a subject between at least 15 minutes and 360 minutes prior to the administration or release of the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat between at least 30 minutes and 360 minutes prior to the administration or release of the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat between at least 60 minutes and 240) minutes prior to the administration or release of the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 90 minutes prior to N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 120 minutes prior to the N-ethyl-2-(5-fluoro-1H-indol-3-yl)-N-methylethan-1-amine form disclosed herein, including those described in Table 5.

In some embodiments, the serotonin receptor modulator is volinanserin, wherein the volinanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 210 minutes prior to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 330 minutes prior to N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is volinanserin, wherein volinanserin is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is ketanserin, wherein the ketanserin is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat between at least 30 minutes and 360 minutes prior to the administration or release of the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat between at least 60 minutes and 240 minutes prior to the administration or release of the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 210 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is ketanserin, wherein ketanserin is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is ritanserin, wherein the ritanserin is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is ritanserin wherein the ritanserin is administered to pretreat between at least 60 minutes and 240 minutes prior to the administration or release of the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 210 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is ritanserin, wherein ritanserin is administered to pretreat between about 60) minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is pimavanserin, wherein the pimavanserin is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat between at least 60 minutes and 240) minutes prior to the administration or release of the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 210 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is pimavanserin, wherein pimavanserin is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is nelotanserin, wherein the nelotanserin is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat between at least 60 minutes and 240) minutes prior to the administration or release of the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 120 minutes prior to the N-ethyl-2-(5-fluoro-1H-indol-3-yl)-N-methylethan-1-amine form disclosed herein, including those described in Table 5.

In some embodiments, the serotonin receptor modulator is nelotanserin, wherein the nelotanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 210 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 270 minutes prior to the N-ethyl-2-(5-fluoro-1H-indol-3-yl)-N-methylethan-1-amine form disclosed herein, including those described in Table 5.

In some embodiments, the serotonin receptor modulator is nelotanserin, wherein the nelotanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is nelotanserin, wherein nelotanserin is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is pruvanserin, wherein the pruvanserin is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat between at least 60 minutes and 240) minutes prior to the administration or release of the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 210 minutes prior to the N-ethyl-2-(5-fluoro-1H-indol-3-yl)-N-methylethan-1-amine form disclosed herein, including those described in Table 5.

In some embodiments, the serotonin receptor modulator is pruvanserin, wherein the pruvanserin is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is pruvanserin, wherein pruvanserin is administered to pretreat between about 60) minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is flibanserin, wherein the flibanserin is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat between at least 60 minutes and 240) minutes prior to the administration or release of the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 210 minutes prior to the N-ethyl-2-(5-fluoro-1H-indol-3-yl)-N-methylethan-1-amine form disclosed herein, including those described in Table 5.

In some embodiments, the serotonin receptor modulator is flibanserin, wherein the flibanserin is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is flibanserin, wherein flibanserin is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is olanzapine, wherein the olanzapine is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat between at least 60 minutes and 240 minutes prior to the administration or release of the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 210 minutes prior to the N-ethyl-2-(5-fluoro-1H-indol-3-yl)-N-methylethan-1-amine form disclosed herein, including those described in Table 5.

In some embodiments, the serotonin receptor modulator is olanzapine, wherein the olanzapine is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is olanzapine, wherein olanzapine is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is risperidone, wherein the risperidone is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat between at least 60 minutes and 240) minutes prior to the administration or release of the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 210 minutes prior to the N-ethyl-2-(5-fluoro-1H-indol-3-yl)-N-methylethan-1-amine form disclosed herein, including those described in Table 5.

In some embodiments, the serotonin receptor modulator is risperidone, wherein the risperidone is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is risperidone, wherein risperidone is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is quetiapine, wherein the quetiapine is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat between at least 60 minutes and 240) minutes prior to the administration or release of the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 150) minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 210 minutes prior to the N-ethyl-2-(5-fluoro-1H-indol-3-yl)-N-methylethan-1-amine form disclosed herein, including those described in Table 5.

In some embodiments, the serotonin receptor modulator is quetiapine, wherein the quetiapine is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is quetiapine, wherein quetiapine is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In some embodiments, the serotonin receptor modulator is buspirone, wherein the buspirone is administered to pretreat at least 15 minutes prior to the administration of the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 30 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat between at least 60 minutes and 240 minutes prior to the administration or release of the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 90 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 120 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 150 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat between about 15 minutes and about 150 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 180 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 210 minutes prior to the N-ethyl-2-(5-fluoro-1H-indol-3-yl)-N-methylethan-1-amine form disclosed herein, including those described in Table 5.

In some embodiments, the serotonin receptor modulator is buspirone, wherein the buspirone is administered to pretreat at least 240 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 270 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 300 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 330 minutes prior to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to pretreat at least 360 minutes prior to the N-ethyl-2-(5-fluoro-1H-indol-3-yl)-N-methylethan-1-amine form disclosed herein, including those described in Table 5. In some preferred embodiments, the serotonin receptor modulator is buspirone, wherein buspirone is administered to pretreat between about 60 minutes and about 180 minutes prior to the administration of the N-ethyl-2-(5-fluoro-1H-indol-3-yl)-N-methylethan-1-amine form disclosed herein, including those described in Table 5.

In certain embodiments, such as those described above a N-ethyl-2-(5-fluoro-1H-indol-3-yl)-N-methylethan-1-amine form disclosed herein, including those described in Table 5, is co-administered with a serotonin receptor modulator in the same or in separate compositions. In one embodiment, the serotonin receptor modulator is administered after the N-ethyl-2-(5-fluoro-1H-indol-3-yl)-N-methylethan-1-amine form disclosed herein, including those described in Table 5. In one embodiment, the N-ethyl-2-(5-fluoro-1H-indol-3-yl)-N-methylethan-1-amine form disclosed herein, including those described in Table 5, is administered in a modified release formulation such that the subject is effectively post-treated with serotonin receptor modulator post to release of an effective amount of the N-ethyl-2-(5-fluoro-1H-indol-3-yl)-N-methylethan-1-amine. In some embodiments, the serotonin receptor modulator is part of a single fixed dose formulation that releases the N-ethyl-2-(5-fluoro-1H-indol-3-yl)-N-methylethan-1-amine first followed by serotonin receptor modulator on two different release profiles. In another embodiment, the N-ethyl-2-(5-fluoro-1H-indol-3-yl)-N-methylethan-1-amine form disclosed herein, including those described in Table 5, is administered first as a single dosage and, after a length of time, serotonin receptor modulator is administered as a second dosage separate from the first dosage. Thus, in some embodiments, the serotonin receptor modulator is administered or released from a composition provided herein post to the administration and/or release of the N-ethyl-2-(5-fluoro-1H-indol-3-yl)-N-methylethan-1-amine form disclosed herein, including those described in Table 5. This allows post-treatment to attenuate activation of the serotonin receptor by the N-ethyl-2-(5-fluoro-1H-indol-3-yl)-N-methylethan-1-amine form disclosed herein, including those described in Table 5.

In some embodiments, the serotonin receptor modulator is administered or released from the composition provided herein to post-treat a subject by at least about at about 5 minutes, 10 minutes, 20 minutes, 30 minutes, 40 minutes, 50 minutes, 1 hour, 1.25 hours, 1.5 hours, 2 hours, or 3 hours post to the release of the N-ethyl-2-(5-fluoro-1H-indol-3-yl)-N-methylethan-1-amine form disclosed herein, including those described in Table 5. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours post to the release of N-ethyl-2-(5-fluoro-1H-indol-3-yl)-N-methylethan-1-amine form disclosed herein, including those described in Table 5. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour post to the release of the N-ethyl-2-(5-fluoro-1H-indol-3-yl)-N-methylethan-1-amine form disclosed herein, including those described in Table 5.

In a preferred embodiment, the serotonin receptor modulator is administered at about 1 hour to about 3 hours post to the administration of the N-ethyl-2-(5-fluoro-1H-indol-3-yl)-N-methylethan-1-amine form disclosed herein, including those described in Table 5.

In some embodiments, the serotonin receptor modulator is eplivanserin, wherein the eplivanserin is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat between at least 30 minutes post and 360 minutes post to the release or administration of the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat between at least 60) minutes post and 360) minutes post to the release or administration the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat between at least 90 minutes and 240 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat at least 120 minutes post to the N-ethyl-2-(5-fluoro-1H-indol-3-yl)-N-methylethan-1-amine form disclosed herein, including those described in Table 5.

In some embodiments, the serotonin receptor modulator is eplivanserin, wherein the eplivanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat at least 210 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat at least 240 minutes post to the N-ethyl-2-(5-fluoro-1H-indol-3-yl)-N-methylethan-1-amine form disclosed herein, including those described in Table 5.

In some embodiments, the serotonin receptor modulator is eplivanserin, wherein the eplivanserin is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is eplivanserin, wherein the eplivanserin is administered to post-treat at least 360 minutes post to administration or release of the N-ethyl-2-(5-fluoro-1H-indol-3-yl)-N-methylethan-1-amine form disclosed herein, including those described in Table 5.

In some preferred embodiments, the serotonin receptor modulator is eplivanserin, wherein eplivanserin is administered to post-treat between about 60 minutes and about 180) minutes post to the administration or release of the N-ethyl-2-(5-fluoro-1H-indol-3-yl)-N-methylethan-1-amine form disclosed herein, including those described in Table 5.

In some embodiments, the serotonin receptor modulator is volinanserin, wherein the volinanserin is administered to post-treat a subject between at least 15 minutes and 360 minutes post to the administration or release of the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat between at least 30 minutes and 360) minutes post to the administration or release of the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 90 minutes post to N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 120 minutes post to the N-ethyl-2-(5-fluoro-1H-indol-3-yl)-N-methylethan-1-amine form disclosed herein, including those described in Table 5.

In some embodiments, the serotonin receptor modulator is volinanserin, wherein the volinanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 210 minutes post to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 270) minutes post to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 330 minutes post to N-ethyl-2-(5-fluoro-1H-indol-3-yl)-N-methylethan-1-amine form disclosed herein, including those described in Table 5. In some embodiments, the serotonin receptor modulator is volinanserin, wherein the volinanserin is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is volinanserin, wherein volinanserin is administered to post-treat between about 60 minutes and about 180) minutes post to the administration of the N-ethyl-2-(5-fluoro-1H-indol-3-yl)-N-methylethan-1-amine form disclosed herein, including those described in Table 5.

In some embodiments, the serotonin receptor modulator is ketanserin, wherein the ketanserin is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat between at least 30 minutes and 360 minutes post to the administration or release of the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 210 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is ketanserin, wherein the ketanserin is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is ketanserin, wherein ketanserin is administered to post-treat between about 60) minutes and about 180 minutes post to the administration of the N-ethyl-2-(5-fluoro-1H-indol-3-yl)-N-methylethan-1-amine form disclosed herein, including those described in Table 5.

In some embodiments, the serotonin receptor modulator is ritanserin, wherein the ritanserin is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is ritanserin wherein the ritanserin is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 210 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is ritanserin, wherein the ritanserin is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is ritanserin, wherein ritanserin is administered to post-treat between about 60 minutes and about 180 minutes post to the administration of the N-ethyl-2-(5-fluoro-1H-indol-3-yl)-N-methylethan-1-amine form disclosed herein, including those described in Table 5.

In some embodiments, the serotonin receptor modulator is pimavanserin, wherein the pimavanserin is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 210 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is pimavanserin, wherein the pimavanserin is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is pimavanserin, wherein pimavanserin is administered to post-treat between about 60) minutes and about 180 minutes post to the administration of the N-ethyl-2-(5-fluoro-1H-indol-3-yl)-N-methylethan-1-amine form disclosed herein, including those described in Table 5.

In some embodiments, the serotonin receptor modulator is nelotanserin, wherein the nelotanserin is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 120 minutes post to the N-ethyl-2-(5-fluoro-1H-indol-3-yl)-N-methylethan-1-amine form disclosed herein, including those described in Table 5.

In some embodiments, the serotonin receptor modulator is nelotanserin, wherein the nelotanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 210 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 270 minutes post to the N-ethyl-2-(5-fluoro-1H-indol-3-yl)-N-methylethan-1-amine form disclosed herein, including those described in Table 5.

In some embodiments, the serotonin receptor modulator is nelotanserin, wherein the nelotanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is nelotanserin, wherein the nelotanserin is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is nelotanserin, wherein nelotanserin is administered to post-treat between about 60 minutes and about 180) minutes post to the administration of the N-ethyl-2-(5-fluoro-1H-indol-3-yl)-N-methylethan-1-amine form disclosed herein, including those described in Table 5.

In some embodiments, the serotonin receptor modulator is pruvanserin, wherein the pruvanserin is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat between at least 60 minutes and 240 minutes post to the administration or release of the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 210 minutes post to the N-ethyl-2-(5-fluoro-1H-indol-3-yl)-N-methylethan-1-amine form disclosed herein, including those described in Table 5.

In some embodiments, the serotonin receptor modulator is pruvanserin, wherein the pruvanserin is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is pruvanserin, wherein the pruvanserin is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is pruvanserin, wherein pruvanserin is administered to post-treat between about 60 minutes and about 180 minutes post to the administration of the N-ethyl-2-(5-fluoro-1H-indol-3-yl)-N-methylethan-1-amine form disclosed herein, including those described in Table 5.

In some embodiments, the serotonin receptor modulator is flibanserin, wherein the flibanserin is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat between about 15 minutes and about 150) minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 210 minutes post to the N-ethyl-2-(5-fluoro-1H-indol-3-yl)-N-methylethan-1-amine form disclosed herein, including those described in Table 5.

In some embodiments, the serotonin receptor modulator is flibanserin, wherein the flibanserin is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is flibanserin, wherein the flibanserin is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is flibanserin, wherein flibanserin is administered to post-treat between about 60) minutes and about 180) minutes post to the administration of the N-ethyl-2-(5-fluoro-1H-indol-3-yl)-N-methylethan-1-amine form disclosed herein, including those described in Table 5.

In some embodiments, the serotonin receptor modulator is olanzapine, wherein the olanzapine is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 210 minutes post to the N-ethyl-2-(5-fluoro-1H-indol-3-yl)-N-methylethan-1-amine form disclosed herein, including those described in Table 5.

In some embodiments, the serotonin receptor modulator is olanzapine, wherein the olanzapine is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is olanzapine, wherein the olanzapine is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is olanzapine, wherein olanzapine is administered to post-treat between about 60 minutes and about 180 minutes post to the administration of the N-ethyl-2-(5-fluoro-1H-indol-3-yl)-N-methylethan-1-amine form disclosed herein, including those described in Table 5.

In some embodiments, the serotonin receptor modulator is quetiapine, wherein the quetiapine is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 210 minutes post to the N-ethyl-2-(5-fluoro-1H-indol-3-yl)-N-methylethan-1-amine form disclosed herein, including those described in Table 5.

In some embodiments, the serotonin receptor modulator is quetiapine, wherein the quetiapine is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is quetiapine, wherein the quetiapine is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is quetiapine, wherein quetiapine is administered to post-treat between about 60 minutes and about 180 minutes post to the administration of the N-ethyl-2-(5-fluoro-1H-indol-3-yl)-N-methylethan-1-amine form disclosed herein, including those described in Table 5.

In some embodiments, the serotonin receptor modulator is risperidone, wherein the risperidone is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat between at least 60 minutes and 240) minutes post to the administration or release of the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 210 minutes post to the N-ethyl-2-(5-fluoro-1H-indol-3-yl)-N-methylethan-1-amine form disclosed herein, including those described in Table 5.

In some embodiments, the serotonin receptor modulator is risperidone, wherein the risperidone is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is risperidone, wherein the risperidone is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is risperidone, wherein risperidone is administered to post-treat between about 60 minutes and about 180 minutes post to the administration of the N-ethyl-2-(5-fluoro-1H-indol-3-yl)-N-methylethan-1-amine form disclosed herein, including those described in Table 5.

In some embodiments, the serotonin receptor modulator is buspirone, wherein the buspirone is administered to post-treat at least 15 minutes post to the administration of the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 30 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat between at least 60 minutes and 240 minutes post to the administration or release of the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 90 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 120 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 150 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat between about 15 minutes and about 150 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 180 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 210 minutes post to the N-ethyl-2-(5-fluoro-1H-indol-3-yl)-N-methylethan-1-amine form disclosed herein, including those described in Table 5.

In some embodiments, the serotonin receptor modulator is buspirone, wherein the buspirone is administered to post-treat at least 240 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 270 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 300 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 330 minutes post to the N-ethyl-2-(5-fluoro-1H-indol-3-yl)-N-methylethan-1-amine form disclosed herein, including those described in Table 5. In some embodiments, the serotonin receptor modulator is buspirone, wherein the buspirone is administered to post-treat at least 360 minutes post to the N-ethyl-2-(5-fluoro-1H-indol-3-yl)-N-methylethan-1-amine form disclosed herein, including those described in Table 5. In some preferred embodiments, the serotonin receptor modulator is buspirone, wherein buspirone is administered to post-treat between about 60 minutes and about 180 minutes post to the administration of the N-ethyl-2-(5-fluoro-1H-indol-3-yl)-N-methylethan-1-amine form disclosed herein, including those described in Table 5.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-HT_{2A} agonists) are used for increasing at least one of translation, transcription, or secretion of neurotrophic factors.

In some embodiments the presently disclosed compounds are given to patients in a low dose that is lower than would produce noticeable psychedelic effects but high enough to provide a therapeutic benefit. This dose range is predicted to be between 200 ug (micrograms) and 2 mg.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with one or more of disclosed compounds can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides methods for increasing neuronal plasticity, comprising contacting a neuronal cell with a compound of the present disclosure. In some embodiments, increasing neuronal plasticity improves a brain disorder described herein.

In some embodiments, a compound of the present disclosure is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present disclosure is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present disclosure is a mouse head-twitch response (HTR) assay.

In some embodiments, the present disclosure provides a method for increasing neuronal plasticity, comprising contacting a neuronal cell with a compound disclosed herein.

### Methods of Treating a Brain Disorder

In some embodiments, the present disclosure provides a method of treating a disease, including administering to a subject in need thereof, a therapeutically effective amount of N-ethyl-2-(5-fluoro-1H-indol-3-yl)-N-methylethan-1-amine of the present disclosure. In some embodiments, the disease is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. In some embodiments, the present disclosure provides a method of treating a brain disorder, including administering to a subject in need thereof, a therapeutically effective amount of a compound of the present disclosure. In some embodiments, the present disclosure provides a method of treating a brain disorder with combination therapy, including administering to a subject in need thereof, a therapeutically effective amount of a compound of the present disclosure and at least one additional therapeutic agent.

In some embodiments, the present salts and solid forms function as 5-HT_{2A} modulators (e.g., 5-HT_{2A} agonists) and are used to treat a brain disorder. In some embodiments, the brain disorders comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, the present disclosure provides a method of treating a brain disorder, comprising administering to a subject in need thereof a therapeutically effective amount of a compound disclosed herein. Thus, in some embodiments, the methods described herein are for treating a disease or disorder that is a brain disease or disorder. In some embodiments, the compounds disclosed herein have, for example, anti-addictive properties, antidepressant properties, anxiolytic properties, or a combination thereof, and thus are useful to treat such disorders. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the methods described herein are for increasing at least one of translation, transcription or secretion of neurotrophic factors. In some embodiments, the compositions provided for use in the present methods herein have, for example, anti-addictive properties, antidepressant properties, anxiolytic properties, or a combination thereof. In some embodiments, the brain disorder is a neuropsychiatric disease. In some embodiments, the methods described herein are for treating a disease or disorder that is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder for example alcohol abuse, opiate addition, or abuse), depression, and anxiety.

In some embodiments, the brain disease or disorder is a neurodegenerative disorder, Alzheimer's disease or Parkinson's disease. In some embodiments, the brain disease or disorder is

In some embodiments, the brain disorder is a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder. In Some embodiments the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the methods described herein are for treating a disease or disorder that is a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, hypoxic brain injury, Chronic traumatic encephalopathy (CTE), traumatic brain injury, dementia, and addiction (e.g., substance use disorder). In some embodiments, the neurological disease is a migraine or cluster headache. In some embodiments, the neurological disease is a neurodegenerative disorder, dementia, Alzheimer's disease, or Parkinson's disease. In some embodiments, the neurological disease is dementia. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety.

In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, the methods described herein are for increasing neuronal plasticity wherein the present compounds have, for example, anti-addictive properties, antidepressant properties, anxiolytic properties, or a combination thereof. In some embodiments, decreased neuronal plasticity is associated with a neuropsychiatric disease. In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments of the method for treating a brain disorder with a salt or solid form disclosed herein, a second therapeutic agent that is an empathogenic agent is administered. Examples of suitable empathogenic agents for use in combination with the present salts and solid forms include phenethylamines, such as MDMA, and analogs thereof.

Other suitable empathogenic agents for use in combination with the presently disclosed compounds include, without limitation,
N-Allyl-3,4-methylenedioxy-amphetamine (MDAL);
N-Butyl-3,4-methylenedioxyamphetamine (MDBU);
N-Benzyl-3,4-methylenedioxyamphetamine (MDBZ);
N-Cyclopropylmethyl-3,4-methy lenedioxy amphetamine (MDCPM);
N,N-Dimethyl-3,4-methylenedioxyamphetamine (MDDM);
N-Ethyl-3,4-methylenedioxyamphetamine (MDE; MDEA);
N-(2-Hydroxyethyl)-3,4-methylenedioxy amphetamine (MDHOET);
N-Isopropyl-3,4-methylenedioxy amphetamine (MDIP);
N-Methyl-3,4-ethylenedioxyamphetamine (MDMC);
N-Methoxy-3,4-methylenedioxyamphetamine (MDMEO);
N-(2-Methoxyethyl)-3,4-methylenedioxyamphetamine (MDMEOET);
alpha,alpha,N-Trimethyl-3,4-methylenedioxyphenethylamine (MDMP; 3,4-Methylenedioxy-N-methylphentermine);
N-Hydroxy-3,4-methylenedioxy amphetamine (MDOH);
3,4-Methylenedioxyphenethylamine (MDPEA);
alpha, alpha-Dimethyl-3,4-methylenedioxyphenethylamine (MDPH; 3,4-methylenedioxy phentermine);
N-Propargyl-3,4-methylenedioxyamphetamine (MDPL);
1,3-Benzodioxolyl-N-methylbutanamine MBDB;
3,4-methylenedioxy-N-methyl-α-ethylphenylethylamine;
3,4-Methylenedioxy amphetamine MDA;
Methylone, also known as 3,4-methylenedioxy-N-methylcathinone;
Ethylone, also known as 3,4-methylenedioxy-N-ethylcathinone;
GHB or Gamma Hydroxy butyrate or sodium oxy bate;
N-Propyl-3,4-methy lenedioxyamphetamine (MDPR); and the like.

In some embodiments, the compounds of the present disclosure are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citalopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present disclosure is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present disclosure provides a method for increasing at least one of translation, transcription or secretion of neurotrophic factors, comprising contacting a neuronal cell with a compound disclosed herein.

### WORKING EXAMPLES

### Example 1

### Synthesis of N-ethyl-2-(5-fluoro-1H-indol-3-yl)-N-methylethan-1-amine

A solution of 4-fluorophenylhydrazine hydrochloride (6.9 mmol) in 4 wt % aqueous H₂SO₄ (10 mL) and DMA (10 mL) is heated at 100° C. under microwave. To this solution is added dihydropyran (6.9 mmol) dropwise over 2 min. Then the reaction is heated at 175° C. (temperature ramp from room temperature to 100° C. over 8 min, then addition of the reagent, heating to 175° C. over 8 min and maintaining this temperature for 30 min, 1000 W). The reaction mixture is cooled to room temperature and extracted two times with EtOAc (25 mL), and the organic layer is washed with water and evaporated. The crude material is purified by column chromatography (eluent CH₂Cl₂/EtOAc 4:1).

A solution of alcohol (5.5 mmol) in CH₂Cl₂ (25 mL) is cooled with an ice bath. To this solution TEA (5.8 mmol) and methanesulfonyl chloride (5.5 mmol) are added. The solution is stirred at room temperature for 2 h. Then it is washed with a saturated solution of NaHCO₃ and H₂O and then dried over Na₂SO₄ and filtered. The solvent is evaporated under reduced pressure. The crude material is purified by flash chromatography (CH₂Cl₂/EtOAc 4:1).

A solution of the mesylate (3.6 mmol) in dioxane (5 mL) is added slowly through an addition funnel to a refluxing solution of the amine (0.26 mol) in dioxane (15 mL) at 100° C. After the addition is completed, the reaction medium is stirred at this temperature for 4 h. After cooling, the mixture is treated with water (20 mL) and extracted with EtOAc (30 mL). The organic layer is dried over Na₂SO₄ and is evaporated to dryness to afford a crude product. The residue is dissolved in 0.1 M HCl. This solution is washed with diethylether and rendered alkaline (pH≈10) with a solution of 1 M NaOH. The mixture is extracted with ether. The organic layer is washed with water, dried over Na₂SO₄, and evaporated to afford the crude base as a solid.

### Example 2

### Salt Screen

N-ethyl-2-(5-fluoro-1H-indol-3-yl)-N-methylethan-1-amine is characterized to evaluate its physical properties. The evaluation is performed by X-ray powder diffraction (XRPD), polarized light microscopy (PLM), differential scanning calorimetry (DSC), thermogravimetry (TG), dynamic vapor sorption/desorption (DVS), and/or solubility testing in organic solvents, water, and mixed solvent systems. XRPD data is used to assess crystallinity. PLM data is used to evaluate crystallinity and particle size/morphology. DSC data is used to evaluate melting point, thermal stability, and crystalline form conversion. TG data is used to evaluate if the free base is a solvate or hydrate, and to evaluate thermal stability. DVS data is used to evaluate hygroscopicity of the free base and if hydrates can be formed at high relative humidity. About 10 to 15 solvents are selected from the list below, based on their properties (polarity, dielectric constant and dipole moment).

TABLE 2
Solvents

| | |
|---|---|
| acetic acid | n-heptane |
| Acetone | n-hexane |
| Acetonitrile | 1,1,1,3,3,3-hexafluoro-2-propanol |
| benzyl alcohol | isobutanol (2-methyl-1-propanol) |
| 1-butanol | isopentanol (3-methyl-1-butanol) |
| 2-butanol | isopropyl alcohol (2-propanol) |
| butyl acetate | isopropylbenzene (cumene) |
| t-butyl methyl ether | Methanol |
| Chlorobenzene | methoxybenzene (anisole) |
| Chloroform | methyl acetate |
| di(ethylene glycol) | methyl ethyl ketone (2-butanone) |
| Dichloromethane | methyl isobutyl ketone |
| diethyl ether | Nitromethane |
| Diethylamine | N-methyl-2-pyrrolidone (NMP) |
| Dimethylacetamide (DMA) | 1-octanol |
| diisopropyl ether | 1-pentanol |
| N,N-dimethyl-formamide (DMF) | 1-propanol |
| dimethyl sulfoxide | Perfluorohexane |
| 1,4-dioxane | propyl acetate |
| 1,2-ethanediol (ethylene glycol) | 1,1,2,2-tetrachloroethane |
| Ethanol | Tetrahydrofuran |
| Ethanolamine | Toluene |
| 2-ethoxyethanol (Cellusolve) | 1,1,1-trichloroethane |
| ethyl acetate | 2,2,2-trifluoroethanol |
| ethyl formate | Water |
| formic acid | o-xylene (1,2-dimethylbenzene) |
| Glycerol | p-xylene (1,4-dimethylbenzene) |

The information obtained is used for designing the subsequent salt screen. The salt screen is performed by reacting the free base with pharmaceutically acceptable acids under various conditions in attempts to generate crystalline salts. Pharmaceutically acceptable acids that may be used are listed below and herein. Specific acids are selected based on the pKa of the free base, and typically 15 to 20 acids are selected. Experiments are performed using 0.5 molar equivalent, 1 molar equivalent and/or 2 molar equivalents of the acid.

TABLE 3
Exemplary Acids

| | |
|---|---|
| naphthalene-1,5-disulfonic acid | citric acid |
| sulfuric acid | d-glucuronic acid |
| ethane-1,2-disulfonic acid | lactobionic acid |
| p-toluenesulfonic acid | D-glucoheptonic acid |
| thiocyanic acid | (−)-L-pyroglutamic acid |
| methanesulfonic acid | L-malic acid |
| dodecylsulfuric acid | hippuric acid |
| naphthalene-2-sulfonic acid | D-gluconic acid |
| benzenesulfonic acid | D,L-lactic acid |
| oxalic acid | oleic acid |
| glycerophosphoric acid | succinic acid |
| ethanesulfonic acid, 2-hydroxy | glutaric acid |
| L-aspartic acid | cinnamic acid |
| maleic acid | adipic acid |
| phosphoric acid | sebacic acid |
| ethanesulfonic acid | (+)-camphoric acid |
| glutamic acid | acetic acid |
| pamoic (embonic) acid | nicotinic acid |
| glutaric acid, 2-oxo- | isobutyric acid |
| 2-naphthoic acid, 1-hydroxy | propionic acid |
| malonic acid | lauric acid |
| gentisic acid | stearic acid |
| L-tartaric acid | orotic acid |
| Hydrochloric acid | carbonic acid |
| galactaric (mucic) acid | Fumaric acid |

Solvent systems for the salt crystallization experiments are selected based on the solubility of the free base and the selected acid. Solvents are used as a single solvent or as solvent mixtures, some containing water. The techniques that are used for salt crystallization are chosen based on the solvent selected and properties of the free base. The following techniques (or combination of techniques) may be used for salt crystallization:
Free base and acid are dissolved in a solvent or mixture of solvents, and the solvents are evaporated at different rates (slow evaporation or fast evaporation) and at different temperatures (ambient or elevated).
Free base and acid are dissolved in a solvent or mixture of solvents (at ambient temperature or an elevated temperature), and the final solution is cooled to a sub-ambient temperature (between −78° C. to 15° C.). The cooling method can be a fast cooling (by plunging the sample into an ice bath or a dry ice/acetone bath), or slow cooling. The solids formed will be recovered by filtration and dried (air dried or vacuum dried).
Free base and acid are dissolved in a solvent or mixture of solvents, and an antisolvent is added to precipitate the salt. The solids formed will be recovered by filtration and dried (air dried or vacuum dried).
Free base and acid are added to a solvent or mixture of solvents, where one or both components are not fully dissolved. The slurry is agitated at different temperatures for a number of days. The solids formed will be recovered by filtration and dried (air dried or vacuum dried). The same experiment can be also performed in solvent systems where the solvents are not miscible.
Free base and acid are milled together (by mechanical milling or by mortar and pestle), with a drop of solvent, or without any solvent.
Free base and acid are melted together, and cooled to various temperatures using various cooling rates.
If an amorphous form of a salt is obtained, the amorphous salt will be exposed to elevated humidity, or elevated temperature (or combination of both), or solvent vapors at various temperatures to form crystalline salts.

The stoichiometric ratio of acid to N-ethyl-2-(5-fluoro-1H-indol-3-yl)-N-methylethan-1-amine is confirmed by 1H NMR, HPLC, or both as is known to those of ordinary skill in the art.

The salts obtained are analyzed by XRPD to determine if they are crystalline and, if so, by DSC to see the melting point and by TG to see if they are hydrated/solvated, and by 1H NMR spectroscopy to ensure chemical integrity. KF water titration is performed on salts that are hydrated. DVS analysis is performed to evaluate hygroscopicity of the salt and if hydrated form is present.

### Example 3

### Polymorph Screen

The active pharmaceutical ingredient (API), which may be a free base or a salt, is characterized to evaluate its physical properties. The evaluation is performed by X-ray powder diffraction (XRPD), polarized light microscopy (PLM), differential scanning calorimetry (DSC), thermogravimetry (TG), dynamic vapor sorption/desorption (DVS), and/or solubility testing in organic solvents, water, and mixed solvent systems. XRPD data is used to assess crystallinity. PLM data is used to evaluate crystallinity and particle size/morphology. DSC data is used to evaluate melting point, thermal stability, and crystalline form conversion. TG data is used to evaluate if the API is a solvate or hydrate, and to evaluate thermal stability. DVS data is used to evaluate hygroscopicity of the API and if hydrates can be formed at high relative humidity. About 10 to 15 solvents may be selected from the list below, based on their properties (polarity, dielectric constant and dipole moment).

TABLE 4
Solvents

| | |
|---|---|
| acetic acid | n-heptane |
| acetone | n-hexane |
| acetonitrile | 1,1,1,3,3,3-hexafluoro-2-propanol |
| benzyl alcohol | isobutanol (2-methyl-1-propanol) |
| 1-butanol | isopentanol (3-methyl-1-butanol) |
| 2-butanol | isopropyl alcohol (2-propanol) |
| butyl acetate | isopropylbenzene (cumene) |
| t-butyl methyl ether | methanol |
| chlorobenzene | methoxybenzene (anisole) |
| chloroform | methyl acetate |
| di(ethylene glycol) | methyl ethyl ketone (2-butanone) |
| dichloromethane | methyl isobutyl ketone |
| diethyl ether | nitromethane |
| diethylamine | N-methyl-2-pyrrolidone (NMP) |
| Dimethylacetamide (DMA) | 1-octanol |
| diisopropyl ether | 1-pentanol |
| N,N-dimethyl-formamide (DMF) | 1-propanol |
| dimethyl sulfoxide | perfluorohexane |
| 1,4-dioxane | propyl acetate |
| 1,2-ethanediol (ethylene glycol) | 1,1,2,2-tetrachloroethane |
| ethanol | tetrahydrofuran |
| ethanolamine | toluene |
| 2-ethoxyethanol (Cellusolve) | 1,1,1-trichloroethane |
| ethyl acetate | 2,2,2-trifluoroethanol |
| ethyl formate | water |
| formic acid | o-xylene (1,2-dimethylbenzene) |
| glycerol | p-xylene (1,4-dimethylbenzene) |

The information obtained is used for designing the subsequent polymorph screen. Solvents are used as a single solvent or as solvent mixtures, some containing water. The techniques used for the polymorph screen are chosen based on the solvent selected and properties of the API. The following techniques (or a combination of techniques) may be used for the polymorph screening:
API is dissolved in a solvent or mixture of solvents, and the solvents are evaporated at different rates (slow evaporation or fast evaporation) and at different temperatures (ambient or elevated).
API is dissolved in a solvent or mixture of solvents (at ambient temperature or an elevated temperature), and the final solution is cooled (between −78° C. to 20° C.). The cooling method can be a fast cooling (by plunging the sample to an ice bath or a dry ice/acetone bath), or slow cooling. The solids formed will be recovered by filtration and dried (air dried or vacuum dried).
API is dissolved in a solvent or mixture of solvents, and an antisolvent is added to precipitate the salt. The solids formed will be recovered by filtration and dried (air dried or vacuum dried).
API is added to a solvent or mixture of solvents, where the API is not fully dissolved. The slurry will be agitated at different temperatures for a number of days. The solids formed will be recovered by filtration and (air dried or vacuum dried).
API is milled (by mechanical milling or by mortar and pestle), with a drop of solvent, or without any solvent.
API is melted and cooled (at different cooling rates, fast and slow, and cooled to different temperatures) to obtain solids.
API is suspended in a solvent or mixture of solvents, and the slurry is placed in a heating/cooling cycle for multiple cycles. The remaining solids after the final cooling cycle will be filtered and (air dried or vacuum dried).
API is processed to obtain an amorphous form (by melting, milling, solvent evaporation, spray drying or lyophilization). The amorphous form will then be exposed to elevated humidity (or elevated temperature, or combination thereof), or to solvent vapors for extended period of days.
API is exposed to elevated humidity (or elevated temperature, or combination thereof), or to solvent vapors for extended period of days.
Two or more polymorphs of the API are mixed in a solvent or solvent systems (some solvent mixtures containing variable amount of water) to obtain a slurry, and the slurry will be agitated (at various temperatures) for an extended period of time (days). The solvent system used can be pre-saturated with the API. The final solids will be filtered and dried (air dried or vacuum dried).
API is heated to a specific temperature and cooled (at ambient conditions or in a dry box).

The solids obtained are analyzed by XRPD to determine if they are crystalline and, if so, by DSC to see the melting point and by TG to see if they are hydrated/solvated, and by 1H NMR spectroscopy to ensure chemical integrity. KF water titration is performed on forms that are hydrated. DVS analysis is performed to evaluate hygroscopicity of the form and if hydrated form is present. In particular variable temperature analyses, including variable temperature XRPD, are performed to assess the stability of each physical form as well as its crystallinity.

Differential scanning calorimetry (DSC) thermograms are obtained using a DSC Q 100 (TA Instruments, New Castle, DE). The temperature axis and cell constant of the DSC cell are calibrated with indium (10 mg, 99.9% pure, melting point 156.6° C., heat of fusion 28.4 J/g). Samples (2.0-5.0 mg) are weighed in aluminum pans on an analytical balance. Aluminum pans without lids are used for the analysis. The samples are equilibrated at 25° C. and heated to 250-300° C. at a heating rate of 10° C./min under continuous nitrogen flow. TG analysis of the samples is performed with a Q 50 (TA Instruments, New Castle, DE). Samples (2.0-5.0 mg) are analyzed in open aluminum pans under a nitrogen flow (50 mL/min) at 25° C. to 210° C. with a heating rate of 10° C./min.

The sample for moisture analysis is allowed to dry at 25° C. for up to 4 hours under a stream of dry nitrogen. The relative humidity is then increased stepwise from 10 to 90% relative humidity (adsorption scan) allowing the sample to equilibrate for a maximum of four hours before weighing and moving on to the next step. The desorption scan is measured from 85 to 0% relative humidity with the same equilibration time. The sample is then dried under a stream of dry nitrogen at 80° C. for 2 hours or until no weight loss is observed.

X-ray powder diffraction data are collected using a Miniflex Tabletop XRD system (Rigaku/MSC, The Woodlands, TX) from 5° to 45 °2θ with steps of 0.1°, and the measuring time is 1.0 second/step. All samples are ground to similar size before exposure to radiation. The powder samples are illuminated using CuKα1 radiation (λ=1.54056 Å) at 30 KV and 15 mA.

Variable temperature XRPD data are collected using a Huber Imaging Plate Guinier Camera 670 employing Ni-filtered CuKα1 radiation (2=1.5405981 Å) produced at 40 kV and 20 mA by a Philips PW 1120/00 generator fitted with a Huber long fine-focus tube PW2273/20 and a Huber Guinier Monochromator Series 611/15. The original powder is packed into a Lindemann capillary (Hilgenberg, Germany) with an internal diameter of 1 mm and a wall thickness of 0.01 mm. The sample is heated at an average rate of 5 Kmin⁻¹ using a Huber High Temperature Controller HTC 9634 unit with the capillary rotation device 670.2. The temperature is held constant at selected intervals for 10 min while the sample is exposed to X-rays and multiple scans are recorded. A 20-range of 4.00-100.0° is used with a step size of 0.005° 20.

In certain embodiments wherein the solid form is a solvate, such as a hydrate, the DSC thermogram reveals endothermic transitions. In accordance with the observed DSC transitions, TGA analysis indicates stages of weight change corresponding to desolvation or dehydration and/or melting of the sample. In the case of hydrates, these results are in harmony with Karl Fisher titration data which indicate the water content of the sample.

The moisture sorption profile of a sample can be generated to assess the stability of a solid form is stable over a range of relative humidities. In certain embodiments, the change in moisture content over 10.0 to 95.0% relative humidity is small. In other embodiments the change in moisture content over 10.0 to 95.0% relative humidity is reversible.

In certain embodiments, the XRPD pattern of a sample of solid form indicates that the sample has a well-defined crystal structure and a high degree of crystallinity.

### Example 4

### Production and Analysis of HCl Salt

N-ethyl-2-(5-fluoro-1H-indol-3-yl)-N-methylethan-1-amine was used to prepare the hydrochloride salt. The HCl salt was then analyzed by XRPD consistent with Example 2. Briefly, following transfer of sample to an Si zero background holder, a preliminary scan in the 20 range of 5-120° was performed to determine the extent of the crystallinity, and the appropriate settings to use for the full scan.

The selected parameters included:
Incident optics: Soller slit=0.04 rad, Programmable Divergence Slit, fixed to 10 mm, Beam mask=20 mm, Anti-scatter slit=2°.
Diffracted optics: Soller slit=0.04 rad, Ni Kβ filter.
2theta range: 5-70°, step size=0.0334°, 1 s step-1.
   The results of the XRPD analysis are illustrated in an XRPD diffractogram provided as FIG. 1 and FIG. 2.
   The solid form of crystalline N-ethyl-2-(5-fluoro-1H-indol-3-yl)-N-methylethan-1-amine HCl analyzed is listed herein, including those described in Table 5.

TABLE 5

| | |
|---|---|
| Solid form of N-ethyl-2-(5-fluoro-1H-indol-3-y1)- | FIG. 1 |
| N-methylethan-1-amine HCl | |

In view of the many possible embodiments to which the principles of the present disclosure may be applied, it should be recognized that the illustrated embodiments are only preferred examples of the disclosure and should not be taken as limiting the scope of the disclosure. Rather, the scope is defined by the following claims. We therefore claim as our invention all that comes within the scope and spirit of these claims.

We claim:

1. A compound, N-ethyl-2-(5-fluoro-1H-indol-3-yl)-N-methylethan-1-amine, wherein the compound is in the form of a salt.

2. A solid form of (i) N-ethyl-2-(5-fluoro-1H-indol-3-yl)-N-methylethan-1-amine or (ii) a N-ethyl-2-(5-fluoro-1H-indol-3-yl)-N-methylethan-1-amine salt.

3. The solid form of claim 2, wherein the N-ethyl-2-(5-fluoro-1H-indol-3-yl)-N-methylethan-1-amine salt is an acid addition salt.

4. The solid form of claim 2, wherein the N-ethyl-2-(5-fluoro-1H-indol-3-yl)-N-methylethan-1-amine salt is a crystalline salt formed from an acid selected from galactaric (mucic) acid, naphthalene-1,5-disulfonic acid, citric acid, sulfuric acid, d-glucuronic acid, ethane-1,2-disulfonic acid, lactobionic acid, p-toluenesulfonic acid, D-glucoheptonic acid, thiocyanic acid, (−)-L-pyroglutamic acid, methanesulfonic acid, L-malic acid, dodecylsulfuric acid, hippuric acid, naphthalene-2-sulfonic acid, D-gluconic acid, benzenesulfonic acid, D,L-lactic acid, oxalic acid, oleic acid, glycerophosphoric acid, succinic acid, ethanesulfonic acid 2-hydroxy, glutaric acid, L-aspartic acid, cinnamic acid, adipic acid, phosphoric acid, sebacic acid, ethanesulfonic acid, (+)-camphoric acid, glutamic acid, acetic acid, fumaric acid, or a combination thereof.

5. The solid form of claim 3, wherein a stoichiometric ratio of acid to N-ethyl-2-(5-fluoro-1H-indol-3-yl)-N-methylethan-1-amine is from 0.4 to 2.2.

6. The solid form of claim 3, wherein a stoichiometric ratio of acid to N-ethyl-2-(5-fluoro-1H-indol-3-yl)-N-methylethan-1-amine is from 0.5 to 2.

7. The solid form of claim 3, wherein a stoichiometric ratio of acid to N-ethyl-2-(5-fluoro-1H-indol-3-yl)-N-methylethan-1-amine is selected from 0.5, 1, or 2.

8. The solid form of claim 2, wherein the solid form is a free base form of N-ethyl-2-(5-fluoro-1H-indol-3-yl)-N-methylethan-1-amine.

9. The solid form of any of claims 2 to 8, wherein the solid form is a hydrate.

10. The solid form of any one of claims 2 to 9, wherein the solid form is a crystalline solid.

11. The solid form of claim 10, wherein the crystalline solid is a substantially single polymorph.

12. The solid form of claim 11, wherein the polymorph is selected to have one or more desired properties.

13. The solid form of claim 12, wherein the one or more desired properties are selected from physical properties, chemical properties, pharmacokinetic properties, or a combination thereof.

14. The solid form of claim 12 or claim 13, wherein the one or more desired properties comprise melting point, glass transition temperature, flowability, thermal stability, shelf life, stability against polymorphic transition, hygroscopic properties, solubility in water and/or organic solvents, reactivity, compatibility with excipients and/or delivery vehicles, bioavailability, absorption, distribution, metabolism, excretion, toxicity including cytotoxicity, dissolution rate, half-life, or a combination thereof.

15. The compound of any of the preceding claims, wherein the solid form of N-ethyl-2-(5-fluoro-1H-indol-3-yl)-N-methylethan-1-amine hydrochloride is a crystalline polymorph of N-ethyl-2-(5-fluoro-1H-indol-3-yl)-N-methylethan-1-amine hydrochloride characterized by two or more, or three XRPD signals selected from the group consisting of 25.1 °2θ, 16.5 °2θ, and 26.2 °2θ (±0.2 °2θ; ±0.1 °2θ; or ±0.0 °2θ; Cu Kα1 radiation).

16. The compound of any of the preceding claims, wherein the solid form of N-ethyl-2-(5-fluoro-1H-indol-3-yl)-N-methylethan-1-amine hydrochloride is a crystalline polymorph of N-ethyl-2-(5-fluoro-1H-indol-3-yl)-N-methylethan-1-amine hydrochloride characterized by signals at 25.1 °2θ, 16.5 °2θ, and 26.2 °2θ (±0.2 °2θ; ±0.1 °2θ; or ±0.0 °2θ; Cu Kα1 radiation).

17. The compound of any of the preceding claims, wherein the solid form of N-ethyl-2-(5-fluoro-1H-indol-3-yl)-N-methylethan-1-amine hydrochloride is a crystalline polymorph of N-ethyl-2-(5-fluoro-1H-indol-3-yl)-N-methylethan-1-amine hydrochloride characterized by two or more, or three or more XRPD signals selected from the group consisting of 25.1 °2θ, 16.5 °2θ, 26.2 °2θ, 15.1 °2θ, and 17.0 °2θ (±0.2 °2θ; ±0.1 °2θ; or ±0.0 °2θ; Cu Kα1 radiation).

18. The compound of any of the preceding claims, wherein the solid form of N-ethyl-2-(5-fluoro-1H-indol-3-yl)-N-methylethan-1-amine hydrochloride is a crystalline polymorph of N-ethyl-2-(5-fluoro-1H-indol-3-yl)-N-methylethan-1-amine hydrochloride characterized by two or more, or three or more XRPD signals selected from the group consisting of 25.1 °2θ, 16.5 °2θ, 26.2 °2θ, 15.1 °2θ, 17.0 °2θ, 11.0 °2θ, and 27.8 °2θ (±0.2 °2θ; ±0.1 °2θ; or ±0.0 °2θ; Cu Kα1 radiation).

19. The compound of any of the preceding claims, wherein the solid form of N-ethyl-2-(5-fluoro-1H-indol-3-yl)-N-methylethan-1-amine hydrochloride is a crystalline polymorph of N-ethyl-2-(5-fluoro-1H-indol-3-yl)-N-methylethan-1-amine hydrochloride characterized by two or more, or three or more XRPD signals selected from the group consisting of 25.1 °2θ, 16.5 °2θ, 26.2 °2θ, 15.1 °2θ, 17.0 °2θ, 11.0 °2θ, 27.8 °2θ, 27.2 °2θ, 31.0 °2θ, and 26.8 °2θ (±0.2 °2θ; ±0.1 °2θ; or ±0.0 °2θ; Cu Kα1 radiation).

20. The compound of any of the preceding claims, wherein the solid form of N-ethyl-2-(5-fluoro-1H-indol-3-yl)-N-methylethan-1-amine hydrochloride is a crystalline polymorph of N-ethyl-2-(5-fluoro-1H-indol-3-yl)-N-methylethan-1-amine hydrochloride characterized by a XRPD diffractogram substantially similar to that shown in FIG. 1.

21. The compound of any of the preceding claims, wherein the solid form of N-ethyl-2-(5-fluoro-1H-indol-3-yl)-N-methylethan-1-amine hydrochloride is a crystalline polymorph of N-ethyl-2-(5-fluoro-1H-indol-3-yl)-N-methylethan-1-amine hydrochloride characterized by any combination of the XRPD peaks set forth in Table 1A.

22. A pharmaceutical composition, comprising the compound of claim 1 or the solid form of N-ethyl-2-(5-fluoro-1H-indol-3-yl)-N-methylethan-1-amine according to any one of claims 2 to 21, and a pharmaceutically acceptable excipient.

23. A method, comprising administering to a subject an effective amount of the compound of claim 1, the solid form of N-ethyl-2-(5-fluoro-1H-indol-3-yl)-N-methylethan-1-amine according to any one of claims 2 to 21, or the pharmaceutical composition according to claim 22.

24. The method of claim 23, wherein the subject has a neurological disease, a psychiatric disorder, or both.

25. The method of claim 24, wherein the neurological disorder is a neurodegenerative disorder.

26. The method of claim 24, wherein the neurological disorder, psychiatric disorder, or both, comprises depression, addiction, anxiety, or a post-traumatic stress disorder.

27. The method of claim 24, wherein the neurological disorder, psychiatric disorder, or both, comprises treatment resistant depression, suicidal ideation, major depressive disorder, bipolar disorder, schizophrenia, or substance use disorder.

28. The method of claim 24, wherein the neurological disorder, psychiatric disorder, or both, comprises stroke, traumatic brain injury, or a combination thereof.

29. The method of any one of claims 23 to 28, wherein administering comprises oral, parenteral, or topical administration.

30. The method of any one of claims 23 to 28, wherein administering comprises oral administration.

31. The method of claim 23, wherein administering comprises administering by injection, inhalation, intraocular, intravaginal, intrarectal or transdermal routes.

32. The method of claim 24, further comprising administering to the subject an effective amount of an empathogenic agent.

33. The method of claim 32, wherein the empathogenic agent is MDMA.

34. The method of claim 24, further comprising administering a 5-HT_{2A} antagonist to the subject.

35. The method of claim 34, wherein the 5-HT_{2A} antagonist is selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, nelotanserin and lorcaserin.

36. A compound, N-ethyl-2-(5-fluoro-1H-indol-3-yl)-N-methylethan-1-amine, in the form of a crystal, a salt, or both.

37. The compound of claim 36, wherein the compound is in the form of a hydrochloride salt.

38. The compound of claim 36 or 37, wherein the compound is in a solid form.

39. The compound of claim 36, wherein the compound is a crystalline form of the free base.

40. The compound of claim 36, wherein the hydrochloride salt form is crystalline.

41. The compound of claim 40, wherein the crystalline salt form is characterized by peaks in an XRPD diffractogram of about 11.0 °2θ+/−0.1 °2θ, 16.5 °2θ+/−0.1 °2θ, and 25.1 °2θ+/−0.1 °2θ.

42. The compound of claim 40, wherein the crystalline salt form is characterized by peaks in the XRPD diffractogram of FIG. 1.
